(19) United States
(12) Patent Application Publication
(10) Pub. No.: US 2010/0227887 A1
(43) Pub. Date: Sep. 9, 2010

(54) PYRIDINE-2-YL CARBOXYLIC ACID AMIDES

(76) Inventors: Georg Jaeschke, Basel (CH); Eric Vieira, Frenkendorf (CH); Juergen Wichmann, Steinen (DE)

Correspondence Address: HOFFMANN-LA ROCHE INC.;PATENT LAW DEPARTMENT, 340 KINGSLAND STREET, NUTLEY, NJ, 07110, US

(21) Appl. No.: 12/711,275

(22) Filed: Feb. 24, 2010

(30) Foreign Application Priority Data
Mar. 5, 2009 (EP) 09154448.6

(51) Int. Cl. A61K 31/4439; C07D 401/12; C07D 417/12; A61K 31/444; A61P 25/00

(52) U.S. Cl. 514/332; 546/275.4; 546/270.7; 546/263; 514/341; 514/342

(57) ABSTRACT

The present invention relates to pyridine-2-yl-carboxylic acid amides which act as metabotropic glutamate receptor antagonists. In particular, the present invention relates to pyridine-2-yl-carboxylic acid amides of formula I

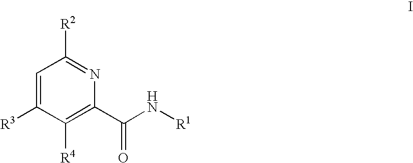

wherein R¹, R², R³, and R⁴ are as described in the specification.

### PRIORITY TO RELATED APPLICATION(S)

This application claims the benefit of European Patent Application No. 09154448.6, filed Mar. 5, 2009, which is hereby incorporated by reference in its entirety.

### BACKGROUND OF THE INVENTION

In the central nervous system (CNS) the transmission of stimuli takes place by the interaction of a neurotransmitter, which is sent out by a neuron, with a neuroreceptor.

Glutamate is the major excitatory neurotransmitter in the brain and plays a unique role in a variety of central nervous system (CNS) functions. The glutamate-dependent stimulus receptors are divided into two main groups. The first main group, namely the ionotropic receptors, forms ligand-controlled ion channels. The metabotropic glutamate receptors (mGluR) belong to the second main group and, furthermore, belong to the family of G-protein coupled receptors.

At present, eight different members of the mGluR family are known and of these, some even have sub-types. According to their sequence homology, signal transduction mechanisms and agonist selectivity, these eight receptors can be sub-divided into three sub-groups:

mGluR1 and mGluR5 belong to group I, mGluR2 and mGluR3 belong to group II and mGluR4, mGluR6, mGluR7 and mGluR8 belong to group III.

Ligands of metabotropic glutamate receptors belonging to group I can be used for the treatment or prevention of acute and/or chronic neurological disorders such as psychosis, epilepsy, schizophrenia, Alzheimer's disease, cognitive disorders and memory deficits, as well as chronic and acute pain.

Other treatable indications in this connection are restricted brain function caused by bypass operations or transplants, poor blood supply to the brain, spinal cord injuries, head injuries, hypoxia caused by pregnancy, cardiac arrest and hypoglycaemia. Further treatable indications are ischemia, Huntington's chorea, amyotrophic lateral sclerosis (ALS), dementia caused by AIDS, eye injuries, retinopathy, idiopathic parkinsonism or parkinsonism caused by medicaments as well as conditions which lead to glutamate-deficiency functions, such as e.g. muscle spasms, convulsions, migraine, urinary incontinence, gastrointestinal reflux disorder, liver damage or failure whether drug or disease induced, Fragile-X syndrome, Down syndrome, autism, nicotine addiction, opiate addiction, anxiety, vomiting, dyskinesia, eating disorders such as bulimia or anorexia nervosa, and depressions.

Disorders mediated full or in part by mGluR5 are for example acute, traumatic and chronic degenerative processes of the nervous system, such as Alzheimer's disease, senile dementia, Parkinson's disease, Huntington's chorea, amyotrophic lateral sclerosis and multiple sclerosis, psychiatric diseases such as schizophrenia and anxiety, depression, pain and drug dependency (Expert Opin. Ther. Patents (2002), 12, (12)).

Selective mGluR5 antagonists are especially useful for the treatment of anxiety and pain.

### SUMMARY OF THE INVENTION

The present invention provides pyridine-2-yl-carboxylic acid amides which act as metabotropic glutamate receptor antagonists.

In particular, the present invention provides pyridine-2-yl-carboxylic acid amides of formula I

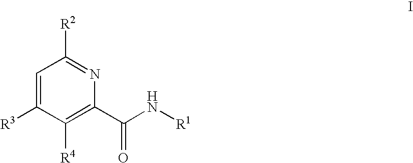

wherein
R¹ is an aromatic 5- or 6-membered ring selected from the group consisting of

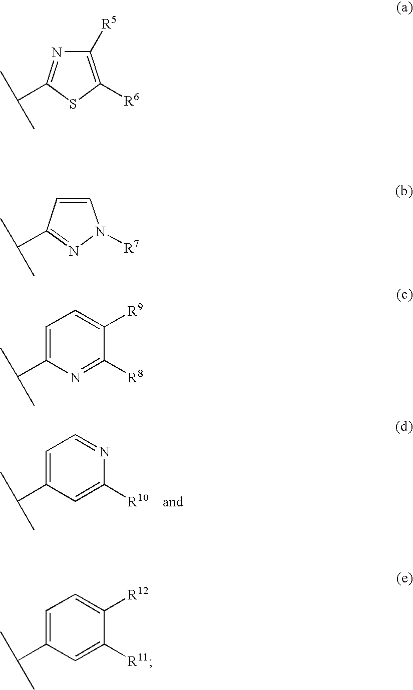

R² is C₁-C₃-alkyl, optionally substituted with one or more OH or halo;
R³ is halo, cyano, or ethynyl;
R⁴ is H or C₁-C₃-alkyl; and
R⁵, R⁶, R⁷, R⁸, R⁹, R¹⁰, R¹¹ and R¹² are each independently H, halo, CN, C₁-C₃-alkyl, C₁-C₃-haloalkyl, C₁-C₃-alkoxy, C₁-C₃-haloalkoxy or OH;
   as well as a pharmaceutically acceptable salt thereof.

Compounds of formula I are metabotropic glutamate receptor antagonists. Compounds of formula I are distinguished by having valuable therapeutic properties. They can be used in the treatment or prevention of mGluR5 receptor mediated disorders.

Present invention provides compounds of formula I and their pharmaceutically acceptable salts, to these compounds as pharmaceutically active substances and to their production.

The invention also provides a process for preparing a compound according to formula I following the general procedures as outlined herein for compounds of formula I.

Moreover the invention provides medicaments containing one or more compounds of the present invention for the treatment and prevention of mGluR5 receptor mediated disorders as outlined above, such as acute and/or chronic neurological disorders, in particular anxiety and chronic or acute pain, urinary incontinence and obesity.

The invention also provides the use of a compound in accordance with the present invention as well as its pharmaceutically acceptable salt for the manufacture of medicaments for the treatment and prevention of mGluR5 receptor mediated disorders as outlined above.

### DETAILED DESCRIPTION OF THE INVENTION

The following definitions of general terms used in the present description apply irrespective of whether the terms in question appear alone or in combination.

The following definitions of the general terms used in the present description apply irrespective of whether the terms in question appear alone or in combination.

As used herein, the term “alkyl” denotes a saturated, i.e. aliphatic hydrocarbon group having a straight or branched carbon chain. Examples for “alkyl” are methyl, ethyl, n-propyl, and isopropyl.

The term “alkoxy” denotes a group —O—R′ wherein R′ is alkyl as defined above.

The term “aromatic” means the presence of an electron sextet in a ring, according to Hückel's rule.

The term “cyano” denotes the group —CN.

The term “ethynyl” denotes the group —C≡CH.

The term “halo” or “halogen” denotes fluoro, chloro, bromo and iodo.

The term “halo-C₁₋₃-alkyl”, “C₁₋₃-haloalkyl” or “C₁₋₃-alkyl substituted with one or more halo” denotes a C₁₋₃-alkyl group as defined above wherein at least one of the hydrogen atoms of the alkyl group is replaced by a halogen atom, preferably fluoro or chloro, most preferably fluoro.

Examples of halo-C₁₋₃-alkyl include methyl, ethyl, propyl, or isopropyl substituted by one or more F, Cl, Br or I atom(s), in particular one, two or three fluoro or chloro, as well as those groups specifically illustrated by the examples herein below. Among the preferred halo-C₁₋₃-alkyl groups are difluoro- or trifluoro-methyl or -ethyl, in particular trifluoromethyl.

The term “C₁₋₃-alkyl substituted with one or more OH” denotes a C₁₋₃-alkyl group as defined above wherein at least one of the hydrogen atoms of the alkyl group is replaced by OH. Preferably, only one hydrogen atom is replaced by OH. Examples are hydroxymethyl or hydroxyethyl, in particular hydroxymethyl.

The term “halo-C₁-C₃-alkoxy”, “C₁-C₃-haloalkoxy” or “C₁₋₃-alkoxy substituted with one or more halo” denotes a C₁₋₃-alkoxy group as defined above wherein at least one of the hydrogen atoms of the alkoxy group is replaced by a halogen atom, preferably fluoro or chloro, most preferably fluoro. Examples of halo-C₁₋₃-alkoxy include methoxy, ethoxy, propoxy, or isopropoxy substituted by one or more F, Cl, Br or I atom(s), in particular one, two or three fluoro or chloro, as well as those groups specifically illustrated by the examples herein below. Among the preferred halo-C₁₋₃-alkoxy groups is trifluoromethoxy.

“Pharmaceutically acceptable,” such as pharmaceutically acceptable carrier, excipient, etc., means pharmacologically acceptable and substantially non-toxic to the subject to which the particular compound is administered.

The term “pharmaceutically acceptable salt” or “pharmaceutically acceptable acid addition salt” embraces salts with inorganic and organic acids, such as hydrochloric acid, nitric acid, sulfuric acid, phosphoric acid, citric acid, formic acid, fumaric acid, maleic acid, acetic acid, succinic acid, tartaric acid, methane-sulfonic acid, p-toluenesulfonic acid and the like.

“Therapeutically effective amount” means an amount that is effective to prevent, alleviate or ameliorate symptoms of disease or prolong the survival of the subject being treated.

In detail, the present invention provides the compound of formula (I)

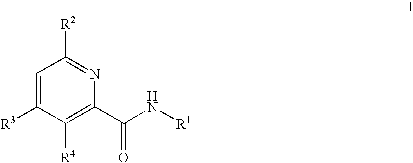

wherein
R¹ is an aromatic 5- or 6-membered ring selected from the group consisting of

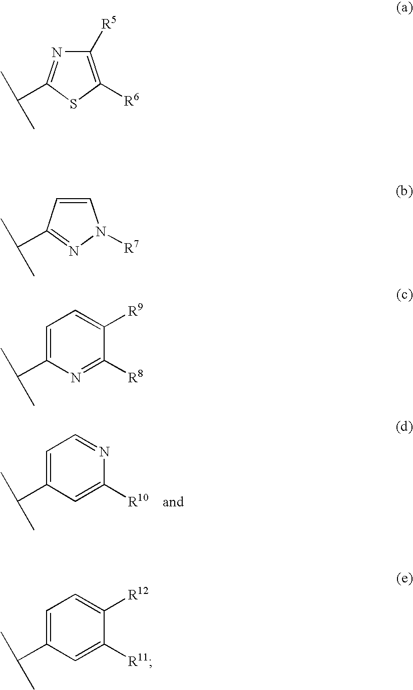

R² is C₁-C₃-alkyl, optionally substituted with one or more OH or halo;
R³ is halo, cyano, or ethynyl;
R⁴ is H or C₁-C₃-alkyl; and
R⁵, R⁶, R⁷, R⁸, R⁹, R¹⁰, R¹¹ and R¹² are each independently H, halo, CN, C₁-C₃-alkyl, C₁-C₃-haloalkyl, C₁-C₃-alkoxy, C₁-C₃-haloalkoxy or OH;
   as well as a pharmaceutically acceptable salt thereof.

It is understood that all embodiments of the invention as described below can be combined with each other.

In certain embodiments, the invention provides a compound of formula (Ia)

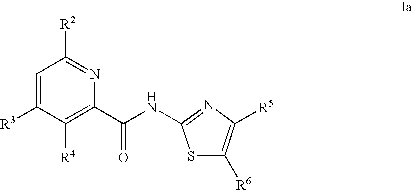

wherein R², R³, R⁴, R⁵ and R⁶ are as described herein, comprising each combination.

In certain embodiments, the invention provides a compound of formula (Ib)

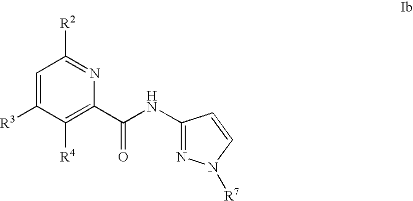

wherein R², R³, R⁴, and R⁷ are as described herein, comprising each combination.

In certain embodiments, the invention provides a compound of formula (Ic)

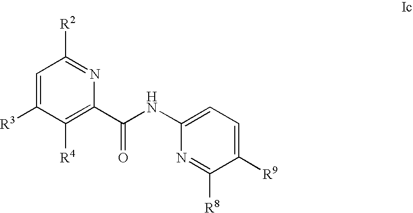

wherein R², R³, R⁴, R⁸ and R⁹ are as described herein.

In certain embodiments, the invention provides a compound of formula (Id)

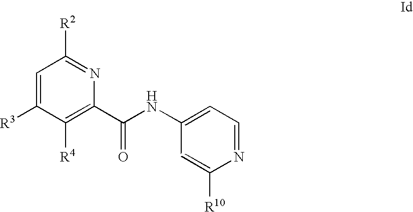

wherein R², R³, R⁴ and R¹⁰ are as described herein, comprising each combination.

In certain embodiments, the invention provides a compound of formula (Ie)

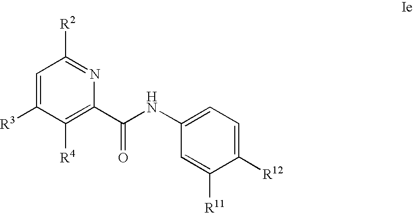

wherein R², R³, R⁴, R¹¹ and R¹² are as described herein, comprising each combination.

In certain embodiments, R¹ is optionally substituted thiazol-2-yl, pyrazol-3-yl, pyridin-2-yl, pyridin-4-yl or phenyl as described in formulae (a), (b), (c), (d) or (e):

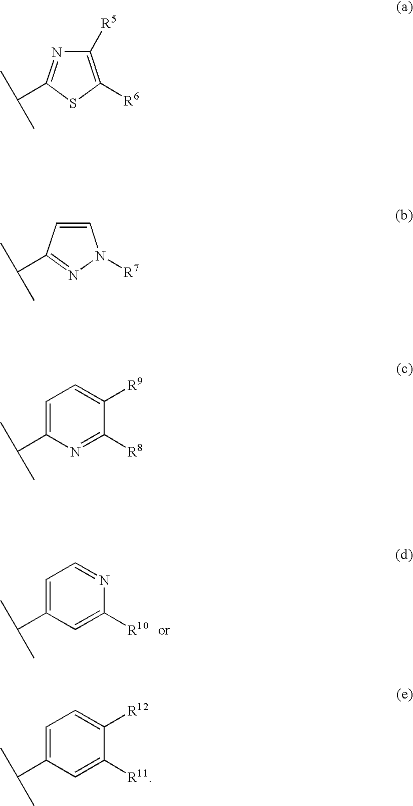

Thereby, the substituents R⁵, R⁶, R⁷, R⁸, R⁹, R¹⁰, R¹¹ and R¹² are each independently H, halo, CN, C₁-C₃-alkyl, C₁-C₃-haloalkyl, C₁-C₃-alkoxy, C₁-C₃-haloalkoxy or OH.

In certain embodiments, the substituents R⁵, R⁶, R⁷, R⁸, R⁹, R¹⁰, R¹¹ and R¹² are each independently H, halo, CN, C₁-C₃-alkyl, C₁-C₃-fluoroalkyl, C₁-C₃-alkoxy, C₁-C₃-fluoroalkoxy or OH.

In certain embodiments, the substituents R⁵, R⁶, R⁷, R⁸, R⁹, R¹⁰, R¹¹ and R¹² are each independently H, fluoro, chloro, cyano, methyl, trifluoromethyl or methoxy.

In certain embodiments, R⁵ is C₁-C₃-alkyl, preferably methyl.

In certain embodiments, R⁶ is H.

In certain embodiments, R⁷ is C₁-C₃-alkyl, preferably methyl.

In certain embodiments, R⁸ is H, halo or C₁-C₃-alkyl. In certain embodiments, R⁸ is H, chloro or methyl.

In certain embodiments, R⁹ is H, halo, cyano or C₁-C₃-alkyl. In certain embodiments, R⁹ is H, fluoro, chloro, cyano or methyl.

In certain embodiments, R¹⁰ is halo or C₁-C₃-alkyl. In certain embodiments, R¹⁰ is fluoro, chloro or methyl.

In certain embodiments, R^{H} is H, halo, C₁-C₃-alkyl, C₁-C₃-haloalkyl, or C₁-C₃-alkoxy. In certain embodiments, R^{H} is H, fluoro, chloro, methyl, trifluoromethyl or methoxy.

In certain embodiments, R¹² is H or halo. In certain embodiments, R¹² is H or fluoro.

In certain embodiments, R² is C₁-C₃-alkyl, optionally substituted with one or more OH or halo.

In certain embodiments, R² is C₁-C₃-alkyl, optionally substituted with one or more OH or fluoro or chloro, preferably OH or fluoro.

In certain embodiments, R² is methyl, ethyl, i-propyl, hydroxymethyl or trifluoromethyl.

In certain embodiments, R³ is halo, cyano, or ethynyl. In certain embodiments, R³ is chloro, bromo, cyano or ethynyl.

In certain embodiments, R⁴ is H or C₁-C₃-alkyl. In certain embodiments, R⁴ is H or methyl. Preferably, R⁴ is H.

In certain embodiments, the invention provides a compound of formula (I)

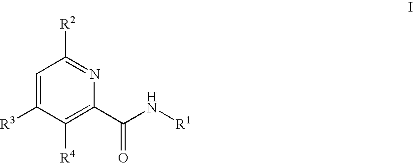

wherein
R¹ is an aromatic 5- or 6-membered ring selected from the group consisting of

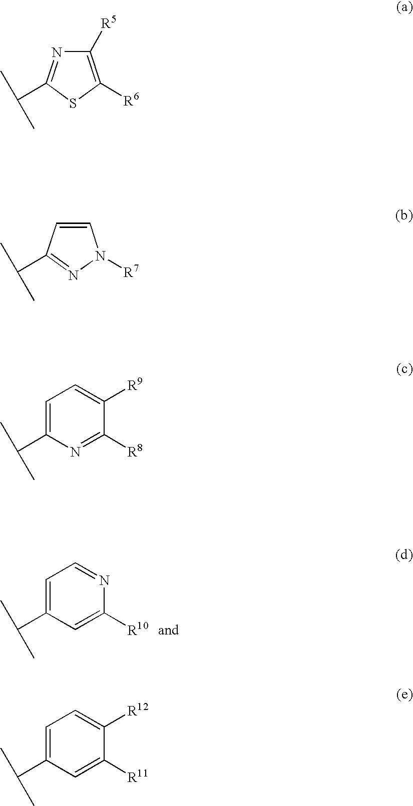

R² is methyl, ethyl, i-propyl, hydroxymethyl or trifluoromethyl;
R³ is chloro, bromo, cyano or ethynyl;

### R⁴ is H; and

R⁵, R⁶, R⁷, R⁸, R⁹, R¹⁰, R¹¹ and R¹² are each independently H, fluoro, chloro, cyano, methyl, trifluoromethyl or methoxy;
   as well as a pharmaceutically acceptable salt thereof.

Examples for the compound according to the invention are shown in the experimental part:

| Ex# | Structure |
|---|---|
| 1 | 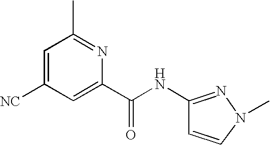 |
| 2 | 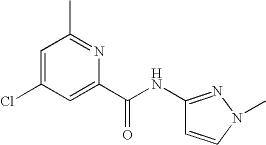 |
| 3 | 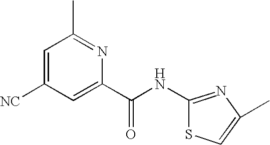 |
| 4 | 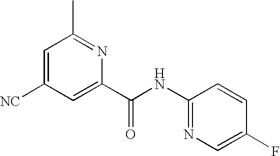 |
| 5 | 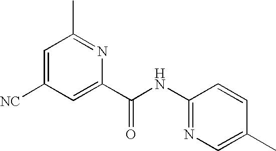 |
| 6 | 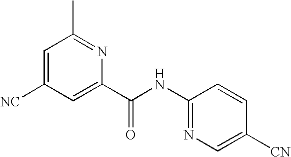 |
| 7 | 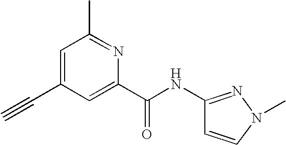 |
| 8 | 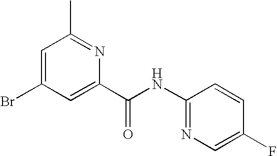 |
| 9 | 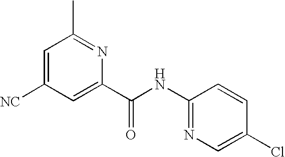 |
| 10 | 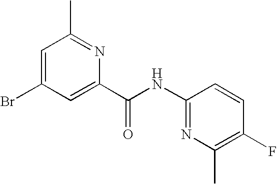 |
| 11 | 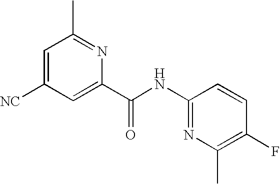 |
| 12 | 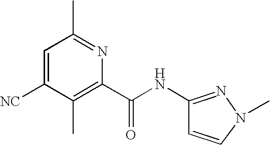 |
| 13 | 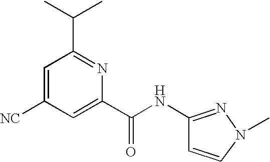 |
| 14 | 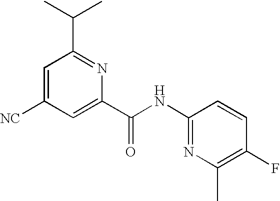 |
| 15 | 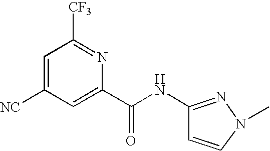 |
| 16 | 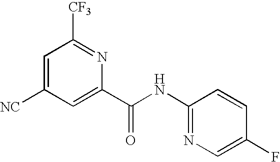 |
| 17 | 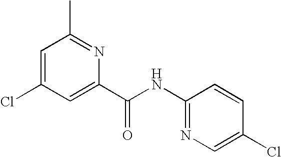 |
| 18 | 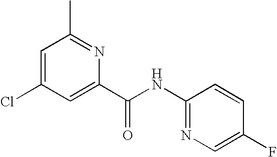 |
| 19 | 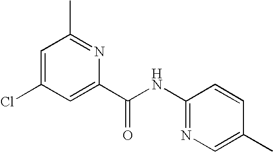 |
| 20 | 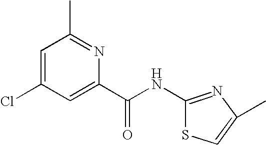 |
| 21 | 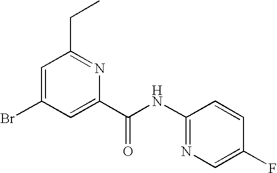 |
| 22 | 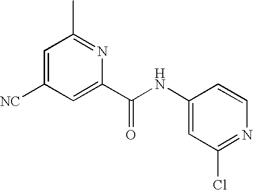 |
| 23 | 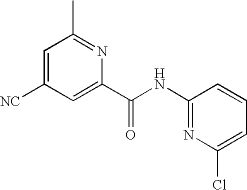 |
| 24 | 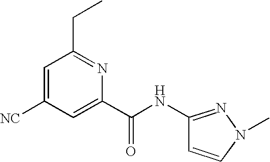 |
| 25 | 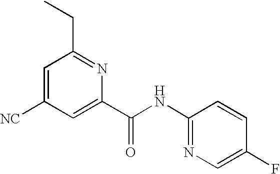 |
| 26 | 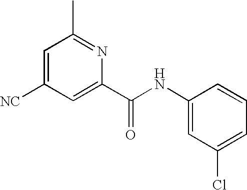 |
| 27 | 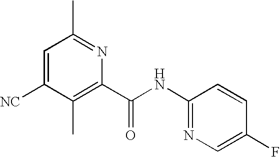 |
| 28 | 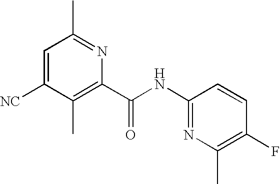 |
| 29 | 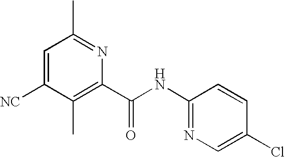 |
| 30 | 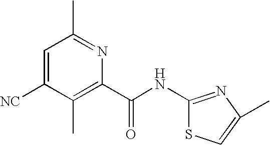 |
| 31 | 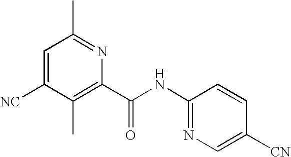 |
| 32 | 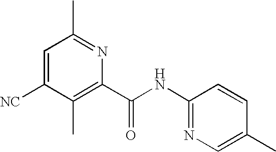 |
| 33 | 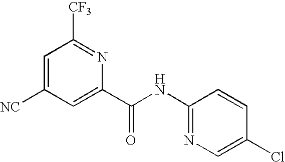 |
| 34 | 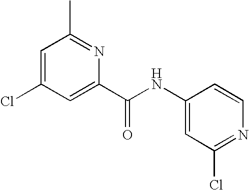 |
| 35 | 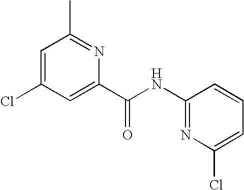 |
| 36 | 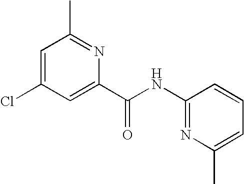 |
| 37 | 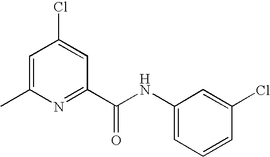 |
| 38 | 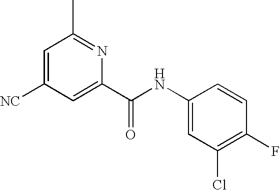 |
| 39 | 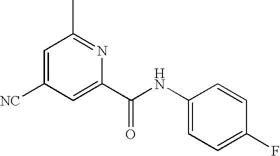 |
| 40 | 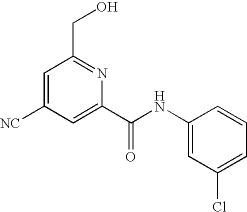 |
| 41 | 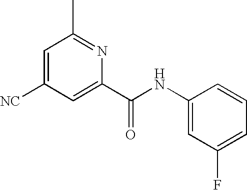 |
| 42 | 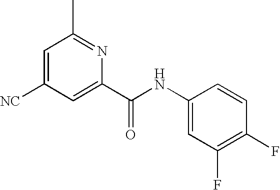 |
| 43 | 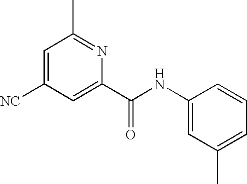 |
| 44 | 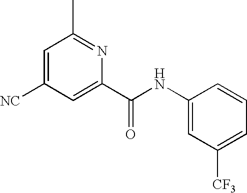 |
| 45 | 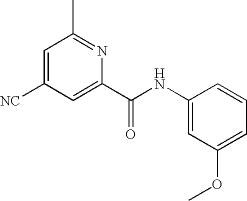 |
| 46 | 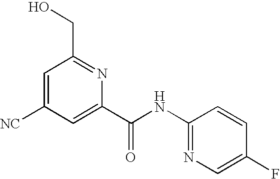 |
| 47 | 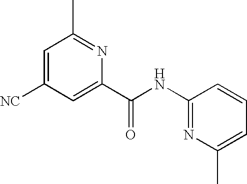 |
| 48 | 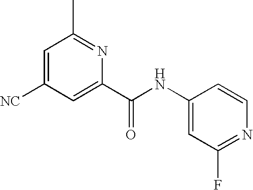 |
| 49 | 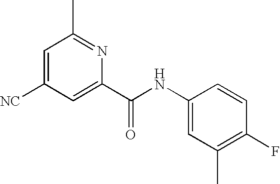 |
| 50 | 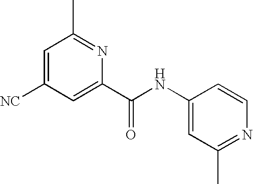 |
| 51 | 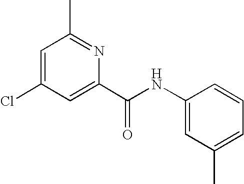 |
| 52 | 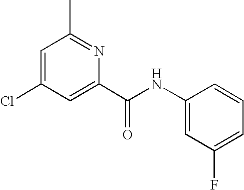 |
| 53 | 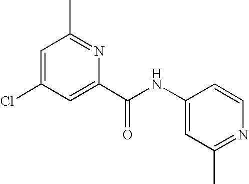 |

Preferred compounds wherein R¹ is thiazol-2-yl (a) are
4-Cyano-6-methyl-pyridine-2-carboxylic acid (4-methyl-thiazol-2-yl)-amide,
4-Chloro-6-methyl-pyridine-2-carboxylic acid (4-methyl-thiazol-2-yl)-amide, or
4-Cyano-3,6-dimethyl-pyridine-2-carboxylic acid (4-methyl-thiazol-2-yl)-amide.

Preferred compounds wherein R¹ is pyrazol-3-yl (b) are those as exemplified in the experimental part. Particularly preferred are
4-Cyano-6-methyl-pyridine-2-carboxylic acid (1-methyl-1H-pyrazol-3-yl)-amide,
4-Chloro-6-methyl-pyridine-2-carboxylic acid (1-methyl-1H-pyrazol-3-yl)-amide,
4-Ethynyl-6-methyl-pyridine-2-carboxylic acid (1-methyl-1H-pyrazol-3-yl)-amide,
4-Cyano-3,6-dimethyl-pyridine-2-carboxylic acid (1-methyl-1H-pyrazol-3-yl)-amide, or
4-Cyano-6-ethyl-pyridine-2-carboxylic acid (1-methyl-1H-pyrazol-3-yl)-amide.

Preferred compounds wherein R¹ is pyridin-2-yl (c) are those as exemplified in the experimental part. Particularly preferred are
4-Cyano-6-methyl-pyridine-2-carboxylic acid (5-fluoro-pyridin-2-yl)-amide,
4-Cyano-6-methyl-pyridine-2-carboxylic acid (5-methyl-pyridin-2-yl)-amide,
4-Cyano-6-methyl-pyridine-2-carboxylic acid (5-cyano-pyridin-2-yl)-amide,
4-Bromo-6-methyl-pyridine-2-carboxylic acid (5-fluoro-pyridin-2-yl)-amide,
4-Cyano-6-methyl-pyridine-2-carboxylic acid (5-chloro-pyridin-2-yl)-amide,
4-Bromo-6-methyl-pyridine-2-carboxylic acid (5-fluoro-6-methyl-pyridin-2-yl)-amide,
4-Cyano-6-methyl-pyridine-2-carboxylic acid (5-fluoro-6-methyl-pyridin-2-yl)-amide,
4-Cyano-6-trifluoromethyl-pyridine-2-carboxylic acid (5-fluoro-pyridin-2-yl)-amide,
4-Chloro-6-methyl-pyridine-2-carboxylic acid (5-fluoro-pyridin-2-yl)-amide,
4-Chloro-6-methyl-pyridine-2-carboxylic acid (5-methyl-pyridin-2-yl)-amide,
4-Cyano-6-methyl-pyridine-2-carboxylic acid (6-chloro-pyridin-2-yl)-amide,
4-Cyano-6-ethyl-pyridine-2-carboxylic acid (5-fluoro-pyridin-2-yl)-amide,
4-Cyano-3,6-dimethyl-pyridine-2-carboxylic acid (5-fluoro-pyridin-2-yl)-amide,
4-Cyano-3,6-dimethyl-pyridine-2-carboxylic acid (5-chloro-pyridin-2-yl)-amide,
4-Chloro-6-methyl-pyridine-2-carboxylic acid (6-chloro-pyridin-2-yl)-amide,
4-Chloro-6-methyl-pyridine-2-carboxylic acid (6-methyl-pyridin-2-yl)-amide,
4-Cyano-6-hydroxymethyl-pyridine-2-carboxylic acid (5-fluoro-pyridin-2-yl)-amide, or
4-Cyano-6-methyl-pyridine-2-carboxylic acid (6-methyl-pyridin-2-yl)-amide.

Preferred compounds wherein R¹ is pyridin-4-yl (d) are those as exemplified in the experimental part. Particularly preferred are
4-Cyano-6-methyl-pyridine-2-carboxylic acid (2-chloro-pyridin-4-yl)-amide,
4-Cyano-6-methyl-pyridine-2-carboxylic acid (2-fluoro-pyridin-4-yl)-amide, or
4-Cyano-6-methyl-pyridine-2-carboxylic acid (2-methyl-pyridin-4-yl)-amide.

Preferred compounds wherein R¹ is phenyl (e) are those as exemplified in the experimental part. Particularly preferred are
4-Cyano-6-methyl-pyridine-2-carboxylic acid (3-chloro-phenyl)-amide,
4-Chloro-6-methyl-pyridine-2-carboxylic acid (3-chloro-phenyl)-amide,
4-Cyano-6-methyl-pyridine-2-carboxylic acid (3-chloro-4-fluoro-phenyl)-amide,
4-Cyano-6-hydroxymethyl-pyridine-2-carboxylic acid (3-chloro-phenyl)-amide,
4-Cyano-6-methyl-pyridine-2-carboxylic acid (3-fluoro-phenyl)-amide,
4-Cyano-6-methyl-pyridine-2-carboxylic acid (3,4-difluoro-phenyl)-amide,
4-Cyano-6-methyl-pyridine-2-carboxylic acid m-tolylamide,
4-Cyano-6-methyl-pyridine-2-carboxylic acid (3-trifluoromethyl-phenyl)-amide,
4-Cyano-6-methyl-pyridine-2-carboxylic acid (3-methoxy-phenyl)-amide,
4-Cyano-6-methyl-pyridine-2-carboxylic acid (4-fluoro-3-methyl-phenyl)-amide, or
4-Chloro-6-methyl-pyridine-2-carboxylic acid m-tolylamide.

The compound of present invention can be obtained by a general synthesis procedure as shown in Scheme I:

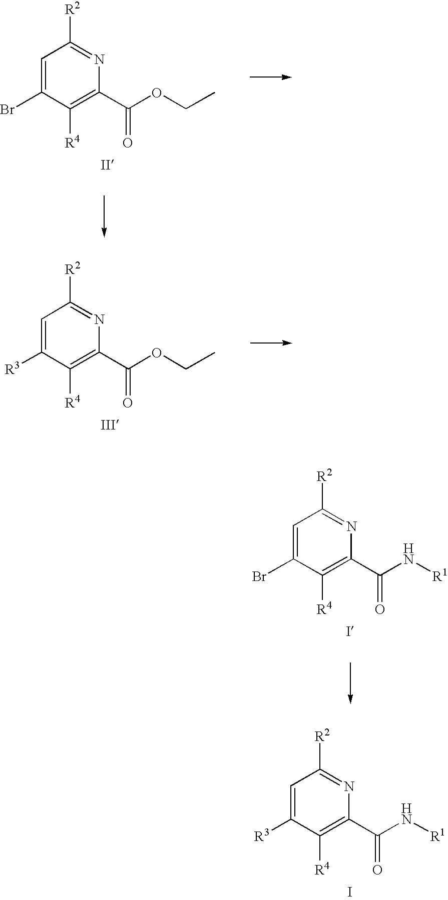

In a certain embodiment, the compound of formula (I) of the invention can be manufactured according to a process comprising the step of reacting a compound of formula (III):

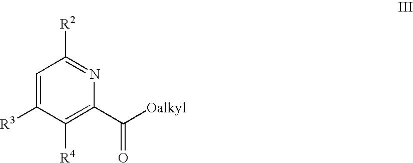

with an amine of formula H₂N—R¹ in the presence of AlMe₃, to obtain a compound of formula (I) wherein R¹, R², R³ and R⁴ are as defined herein above and alkyl is preferably methyl, ethyl or propyl, in particular ethyl.

In a certain embodiment, the compound of formula (I) of the present invention can be manufactured according to a process comprising the step of reacting a compound of formula (I′):

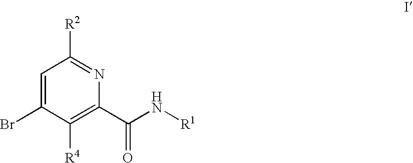

a) either with fuming HCl and NaCl in an organic solvent to obtain a compound of formula (I) wherein R¹, R², and R⁴ are as defined herein above, and R³ is Cl;
b) or with zinkcyanid and tetrakis-(triphenylphosphine)-palladium(0) in an organic solvent, using microwave, to obtain a compound of formula (I) wherein R¹, R², and R⁴ are as defined herein above, and R³ is CN;
c) or with triethylamine, triphenylphosphine and PdCl₂(PPh₃)₂, followed by the addition of copper(I) iodide and ethynyltrimethylsilane, to obtain a compound of formula (I) wherein R¹, R², and R⁴ are as defined herein above, and R³ is ethynyl.

The synthesis of the specific examples is shown in the experimental part.

Present invention further provides the compound of formula I, produced by the methods as described herein.

Present invention further provides a pharmaceutical composition/medicament comprising at least one of the compounds according to formula I as described herein as well as its pharmaceutically acceptable salt. The pharmaceutical composition can at least contain one pharmaceutical excipient and/or carrier. Thereby, the pharmaceutical composition/medicament is preferably used for the treatment and prevention of mGluR5 receptor mediated disorders, and in particular for the indications as described herein.

Present invention further provides a compound of formula I as described herein as well as its pharmaceutically acceptable salt for the use as a medicament, in particular for the use as a medicament for the treatment and prevention of mGluR5 receptor mediated disorders. Preferably, the compound of formula I as described herein as well as its pharmaceutically acceptable salt is used in the treatment or prevention of psychosis, epilepsy, schizophrenia, Alzheimer's disease, cognitive disorders and memory deficits, chronic and acute pain, restricted brain function caused by bypass operations or transplants, poor blood supply to the brain, spinal cord injuries, head injuries, hypoxia caused by pregnancy, cardiac arrest and hypoglycaemia, ischemia, Huntington's chorea, amyotrophic lateral sclerosis (ALS), dementia caused by AIDS, eye injuries, retinopathy, idiopathic parkinsonism or parkinsonism caused by medicaments, muscle spasms, convulsions, migraine, urinary incontinence, gastrointestinal reflux disorder, liver damage or failure whether drug or disease induced, Fragile-X syndrome, Down syndrome, autism, nicotine addiction, opiate addiction, anxiety, vomiting, dyskinesia, eating disorders, in particular bulimia or anorexia nervosa, and depressions, particularly for the treatment and prevention of acute and/or chronic neurological disorders, anxiety, the treatment of chronic and acute pain, urinary incontinence and obesity.

Present invention further provides the use of a compound of formula I as described herein, as well as its pharmaceutically acceptable salt, for the manufacture of a medicament, preferably for the treatment and prevention of mGluR5 receptor mediated disorders, and in particular for the indications as described herein.

### Biological Assay and Data

The pharmacological activity of the compounds was tested using the following method: For binding experiments, cDNA encoding human mGlu 5a receptor was transiently transfected into EBNA cells using a procedure described by Schlaeger and Christensen [Cytotechnology 15:1-13 (1998)]. Cell membrane homogenates were stored at −80° C. until the day of assay where upon they were thawed and resuspended and polytronised in 15 mM Tris-HCl, 120 mM NaCl, 100 mM KCl, 25 mM CaCl₂, 25 mM MgCl₂ binding buffer at pH 7.4 to a final assay concentration of 20 μg protein/well.

Saturation isotherms were determined by addition of twelve [³H]MPEP concentrations (0.04-100 nM) to these membranes (in a total volume of 200 μl) for 1 h at 4° C. Competition experiments were performed with a fixed concentration of [³H]MPEP (2 nM) and IC₅₀ values of test compounds evaluated using 11 concentrations (0.3-10,000 nM). Incubations were performed for 1 h at 4° C.

At the end of the incubation, membranes were filtered onto unifilter (96-well white microplate with bonded GF/C filter preincubated 1 h in 0.1% PEI in wash buffer, Packard Bio-Science, Meriden, Conn.) with a Filtermate 96 harvester (Packard BioScience) and washed 3 times with cold 50 mM Tris-HCl, pH 7.4 buffer. Nonspecific binding was measured in the presence of 10 μM MPEP. The radioactivity on the filter was counted (3 min) on a Packard Top-count microplate scintillation counter with quenching correction after addition of 45 μl of microscint 40 (Canberra Packard S. A., Zürich, Switzerland) and shaking for 20 min.

For functional assays, [Ca²⁺]i measurements were performed as described previously by Porter et al. [Br. J. Pharmacol. 128:13-20 (1999)] on recombinant human mGlu 5a receptors in HEK-293 cells. The cells were dye loaded using Fluo 4-AM (obtainable by FLUKA, 0.2 μM final concentration). [Ca²⁺]i measurements were performed using a fluorometric imaging plate reader (FLIPR, Molecular Devices Corporation, La Jolla, Calif., USA). Antagonist evaluation was performed following a 5 min preincubation with the test compounds followed by the addition of a submaximal addition of agonist.

The inhibition (antagonists) curves were fitted with a four parameter logistic equation giving IC₅₀, and Hill coefficient using iterative non linear curve fitting software (Xcel fit).

For binding experiments the Ki values of the compounds tested are given. The Ki value is defined by the following formula:

Kᵢ=IC₅₀/[1+L/K_{d}]

in which the IC₅₀ values are those concentrations of the compounds tested which cause 50% inhibition of the competing radioligand ([³H]MPEP). L is the concentration of radioligand used in the binding experiment and the K_{d} value of the radioligand is empirically determined for each batch of membranes prepared.

The compounds of the present invention are mGluR 5a receptor antagonists. The activities of compounds of formula I as measured in the assay described above are in the range of Kᵢ<4 μM and preferably <150 nM.

| | Ki |
|---|---|
| Ex# | (nM) |
| 1 | 38 |
| 2 | 73 |
| 3 | 15 |
| 4 | 6 |
| 5 | 11 |
| 6 | 109 |
| 7 | 33 |
| 8 | 37 |
| 9 | 8 |
| 10 | 61 |
| 11 | 57 |
| 12 | 143 |
| 16 | 139 |
| 18 | 30 |
| 19 | 109 |
| 20 | 14 |
| 22 | 25 |
| 23 | 8 |
| 24 | 36 |
| 25 | 11 |
| 26 | 6 |
| 27 | 31 |
| 29 | 97 |
| 30 | 37 |
| 35 | 34 |
| 36 | 15 |
| 37 | 55 |
| 38 | 27 |
| 40 | 44 |
| 41 | 9 |
| 42 | 117 |
| 43 | 9 |
| 44 | 42 |
| 45 | 57 |
| 46 | 111 |
| 47 | 18 |
| 48 | 33 |
| 49 | 72 |
| 50 | 99 |
| 51 | 97 |

The present invention also provides pharmaceutical compositions containing compounds of the invention, for example, compounds of formula I or pharmaceutically acceptable salts thereof and a pharmaceutically acceptable carrier. Such pharmaceutical compositions can be in the form of tablets, coated tablets, dragées, hard and soft gelatin capsules, solutions, emulsions or suspensions. The pharmaceutical compositions also can be in the form of suppositories or injectable solutions.

The pharmaceutical compositions of the invention, in addition to one or more compounds of the invention, contain a pharmaceutically acceptable carrier. Suitable pharmaceutically acceptable carriers include pharmaceutically inert, inorganic or organic carriers. gelatinLactose, corn starch or derivatives thereof, talc, stearic acid or its salts and the like can be used, for example, as such carriers for tablets, coated tablets, dragées and hard gelatin capsules. Suitable carriers for soft gelatin capsules are, for example, vegetable oils, waxes, fats, semi-solid and liquid polyols and the like; depending on the nature of the active substance no carriers are, however, usually required in the case of soft gelatin capsules. Suitable carriers for the production of solutions and syrups are, for example, water, polyols, sucrose, invert sugar, glucose and the like. Adjuvants, such as alcohols, polyols, glycerol, vegetable oils and the like, can be used for aqueous injection solutions of water-soluble salts of compounds of formula (I), but as a rule are not necessary. Suitable carriers for suppositories are, for example, natural or hardened oils, waxes, fats, semi-liquid or liquid polyols and the like.

In addition, the pharmaceutical compositions can contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

As mentioned earlier, medicaments containing a compound of formula (I) or pharmaceutically acceptable salts thereof and a therapeutically inert excipient are also an object of the present invention, as is a process for the production of such medicaments which comprises bringing one or more compounds of formula I or pharmaceutically acceptable salts thereof and, if desired, one or more other therapeutically valuable substances into a galenical dosage form together with one or more therapeutically inert carriers.

As further mentioned earlier, the use of the compounds of formula (I) for the preparation of medicaments useful in the prevention and/or the treatment of the above recited diseases is also an object of the present invention.

The dosage at which compounds of the invention can be administered can vary within wide limits and will, of course, be fitted to the individual requirements in each particular case. In general, the effective dosage for oral or parenteral administration is between 0.01-20 mg/kg/day, with a dosage of 0.1-10 mg/kg/day being preferred for all of the indications described. The daily dosage for an adult human being weighing 70 kg accordingly lies between 0.7-1400 mg per day, preferably between 7 and 700 mg per day.

### Preparation of Pharmaceutical Compositions Comprising Compounds of the Invention

### Example I

Tablets of the following composition are produced in a conventional manner:

| | mg/Tablet |
|---|---|
| Active ingredient | 100 |
| Powdered. lactose | 95 |
| White corn starch | 35 |
| Polyvinylpyrrolidone | 8 |
| Na carboxymethylstarch | 10 |
| Magnesium stearate | 2 |
| Tablet weight | 250 |

### Example II

Tablets of the following composition are produced in a conventional manner:

| | mg/Tablet |
|---|---|
| Active ingredient | 200 |
| Powdered. lactose | 100 |
| White corn starch | 64 |
| Polyvinylpyrrolidone | 12 |
| Na carboxymethylstarch | 20 |
| Magnesium stearate | 4 |
| Tablet weight | 400 |

### Example III

Capsules of the following composition are produced:

| | mg/Capsule |
|---|---|
| Active ingredient | 50 |
| Crystalline. lactose | 60 |
| Microcrystalline cellulose | 34 |
| Talc | 5 |
| Magnesium stearate | 1 |
| Capsule fill weight | 150 |

The active ingredient having a suitable particle size, the crystalline lactose and the microcrystalline cellulose are homogeneously mixed with one another, sieved and thereafter talc and magnesium stearate are admixed. The final mixture is filled into hard gelatin capsules of suitable size.

The following examples are provided to further elucidate the invention and are not intended to limit the invention to the sole compounds exemplified:

### Intermediates

### Intermediate 1: 4-Cyano-6-methyl-pyridine-2-carboxylic acid ethyl ester

A mixture of 4-bromo-6-methyl-pyridine-2-carboxylic acid ethyl ester [CAS No. 947179-03-5] (1.22 g, 5.0 mmol), zinkcyanid (0.88 g, 7.0 mmol) and tetrakis-(triphenylphosphine)-palladium(0) (578 mg, 0.5 mmol) in DMF (15 ml) was stirred in a microwave oven for 15 minutes at 160° C. The mixture was poured into water (50 ml) and extracted with ethyl acetate (2×50 ml). The combined organic layers were washed with brine (50 ml) and dried (MgSO₄). Removal of the solvent left a light yellow solid (1.74 g), which was further purified by flash chromatography on silica gel [heptan/ethyl acetate (20-80%)] to yield the title compound as a white solid (0.48 g, 50%), MS (ISP) m/e=191.2 [(M+H)⁺], mp 50° C.

### Intermediate 2: 4-Bromo-6-isopropyl-pyridine-2-carboxylic acid ethyl ester

### Step A

A stirred solution of 2-isopropyl-4-nitro-pyridine-1-oxide [CAS No. 113548-79-1] (3.2 g, 17.6 mmol) and acetyl bromide (10.8 g, 87.8 mmol) in acetic acid (13 ml) was heated under reflux conditions for 90 min, evaporated, 2N sodium carbonate/ice (50 ml) was added and the mixture was extracted with dichloromethane (2×80 ml). The combined organic layers were washed with brine (50 ml), dried (MgSO₄) and evaporated to yield 4-bromo-2-isopropyl-pyridine-1-oxide (3.77 g, 99%) as a brown oil, MS (ISP) m/e=218.2 [(M+H)⁺].

### Step B

A stirred mixture of 4-bromo-2-isopropyl-pyridine-1-oxide (3.77 g, 17.4 mmol), triethylamine (7.06 g, 69.8 mmol), trimethylsilylcyanide (5.19 g, 52.3 mmol) i and acetonitrile (29 ml) was heated under reflux conditions for 17 h, evaporated, water (30 ml) was added and the mixture was extracted with diethyl ether (2×50 ml). The combined organic layers were washed with water (30 ml) and brine (30 ml), dried (MgSO₄) and evaporated. The crude product was further purified by flash chromatography on silica gel (heptane/ethylacetate (0-25%) to yield 4-bromo-2-cyano-6-isopropyl-pyridine (0.87 g, 22%) as a light yellow oil, MS (ISP) m/e=227.1 [(M+H)⁺].

### Step C

A stirred mixture of 4-bromo-2-cyano-6-isopropyl-pyridine (0.86 g, 3.82 mmol) and 80% sulfuric acid (2.65 g, 21.6 mmol) was heated for 1 h at 130° C., cooled to room temperature, and ethanol (35 ml) was added. The mixture was heated under efflux conditions for 2 h, evaporated, brine (30 ml) was added and the mixture was extracted with ethyl acetate (2×40 ml). The combined organic layers were washed with brine (25 ml), dried (MgSO₄) and evaporated. The crude product was further purified by flash chromatography on silica gel (heptane/ethylacetate (0-30%) to yield the title compound (0.63 g, 60%) as a colorless oil, MS (ISP) m/e=274.2 [(M+H)⁺].

### Intermediate 3: 4-Bromo-6-trifluoromethyl-pyridine-2-carboxylic acid ethyl ester

### Step A

Reaction of 4-nitro-2-trifluoromethyl-pyridine-1-oxide [CAS No. 147149-97-1] and acetyl bromide according to the general method of Intermediate 2 (step A) yielded 4-bromo-2-trifluoromethyl-pyridine-1-oxide as an orange solid, MS (ISP) m/e=244.2 [(M+H)⁺].

### Step B

Reaction of 4-bromo-2-trifluoromethyl-pyridine-1-oxide and trimethylsilylcyanide according to the general method of Intermediate 2 (step B) yielded 4-bromo-2-cyano-6-trifluoromethyl-pyridine as a yellow solid, MS (EI) m/e=250.0 [(M)⁺], mp 88.5° C.

### Step C

Transformation of 4-bromo-2-cyano-6-trifluoromethyl-pyridine according to the general method of Intermediate 2 (step C) yielded the title compound as yellow solid, MS (ISP) m/e=298.2 [(M+H)⁺], mp 51.5° C.

### Intermediate 4: 4-Bromo-6-ethyl-pyridine-2-carboxylic acid ethyl ester

### Step A

Reaction of 4-bromo-2-ethyl-pyridine-1-oxide [CAS No. 18880-09-06] and trimethylsilylcyanide according to the general method of Intermediate 2 (step B) yielded 4-bromo-2-cyano-6-ethyl-pyridine as a yellow oil, MS (ISP) m/e=213.1 [(M+H)⁺].

### Step B

Transformation of 4-bromo-2-cyano-6-ethyl-pyridine according to the general method of Intermediate 2 (step C) yielded the title compound as a light yellow oil, MS (ISP) m/e=260.1 [(M+H)⁺].

### Intermediate 5: 4-Cyano-3,6-dimethyl-pyridine-2-carboxylic acid ethyl ester

The title compound, light yellow solid, MS (ISP) m/e=205.2 [(M+H)⁺], mp 55.5° C., was prepared in accordance with the general method of Intermediate 1 from 4-bromo-3,6-dimethylpyridine-2-carboxylic acid ethyl ester [CAS No. 947179-26-2].

### Intermediate 6: 4-Cyano-6-trifluoromethyl-pyridine-2-carboxylic acid ethyl ester

The title compound, yellow solid, MS (ISP) m/e=245.1 [(M+H)⁺], mp 71.5° C., was prepared in accordance with the general method of Intermediate 1 from 4-bromo-6-trifluoromethyl-pyridine-2-carboxylic acid ethyl ester (Intermediate 3).

### Intermediate 7: 4-Cyano-6-hydroxymethyl-pyridine-2-carboxylic acid ethyl ester

The title compound, light brown solid, MS (ISP) m/e=207.1 [(M+H)⁺], mp 87.5° C., was prepared in accordance with the general method of Intermediate 1 from 4-bromo-6-hydroxymethyl-pyridine-2-carboxylic acid ethyl ester [CAS No. 1001415-17-3].

### EXAMPLES

### Example 1

### 4-Cyano-6-methyl-pyridine-2-carboxylic acid (1-methyl-1H-pyrazol-3-yl)-amide

The title compound, white solid, MS (ISP) m/e=242.2 [(M+H)⁺], mp 201° C., was prepared in accordance with the general method of Intermediate 1 from 4-bromo-6-methylpyridine-2-carboxylic acid (1-methyl-1H-pyrazol-3-yl)-amide [CAS No. 947179-05-7].

### Example 2

### 4-Chloro-6-methyl-pyridine-2-carboxylic acid (1-methyl-1H-pyrazol-3-yl)-amide

To a stirred mixture of 4-bromo-6-methyl-pyridine-2-carboxylic acid (1-methyl-1H-pyrazol-3-yl)-amide [CAS No. 947179-05-7] (120 mg, 0.4 mmol) in 2-butanone (2.5 ml) was added sodium chloride (88 mg, 1.5 mmol) and fuming (37%) hydrochloric acid (0.03 ml, 1 mmol). The mixture was heated under reflux conditions for 72 h. Additional sodium chloride (88 mg) and hydrochloric acid (0.03 ml) were added after 24 h and 48 h, respectively. The mixture was added to 50 ml saturated NaHCO₃-solution and ice (50 ml) and extracted with ethyl acetate (2×50 ml). The combined organic layers were washed with brine (50 ml) and dried (MgSO₄). Removal of the solvent left a light yellow solid, which was purified by crystallization from dichloromethane/heptane to yield the title compound as an off-white solid (81 mg, 79%), MS (ISP) m/e=251.2 [(M+H)⁺], mp 203.5° C.

### Example 3

### 4-Cyano-6-methyl-pyridine-2-carboxylic acid (4-methyl-thiazol-2-yl)-amide

To a cooled (water) and stirred solution of commercially available 2-amino-4-methylthiazole (126 mg, 1.1 mmol) in dioxane (2 ml) was added drop wise a 2M trimethylaluminium solution in heptane (0.55 ml, 1.1 mmol). The solution was allowed to stir for 1 h at room temperature. Afterwards a solution of 4-cyano-6-methyl-pyridine-2-carboxylic acid ethyl ester (intermediate 1) (0.07 g, 0.37 mmol) in dioxane (0.6 ml) was added and the reaction mixture was allowed to stir for 2 h at 110° C. The mixture was poured into potassium-sodium-tartrate solution and ice (50 ml) and extracted with ethyl acetate (2×50 ml). The combined organic layers were washed with brine (50 ml) and dried (MgSO₄). Removal of the solvent left the crude product (0.21 g) as a yellow solid, which was further purified by flash chromatography on silica gel [heptan/ethyl acetate (20-100%)] to yield the title compound as a yellow solid (70 mg, 74%), MS (ISP) m/e=259.1 [(M+H)⁺], mp 211° C.

### Example 4

### 4-Cyano-6-methyl-pyridine-2-carboxylic acid (5-fluoro-pyridin-2-yl)-amide

The title compound, off-white solid, MS (ISP) m/e=257.3 [(M+H)⁺], mp 204.5° C., was prepared from 4-cyano-6-methyl-pyridine-2-carboxylic acid ethyl ester (Intermediate 1) and commercially available 2-amino-5-fluoropyridine according to the general method of Example 3.

### Example 5

### 4-Cyano-6-methyl-pyridine-2-carboxylic acid (5-methyl-pyridin-2-yl)-amide

The title compound, light yellow solid, MS (ISP) m/e=253.1 [(M+H)⁺], mp 196° C., was prepared from 4-cyano-6-methyl-pyridine-2-carboxylic acid ethyl ester (Intermediate 1) and commercially available 6-amino-3-picoline according to the general method of Example 3.

### Example 6

### 4-Cyano-6-methyl-pyridine-2-carboxylic acid (5-cyano-pyridin-2-yl)-amide

The title compound, off-white solid, MS (ISP) m/e=264.1 [(M+H)⁺], mp 279° C., was prepared from 4-cyano-6-methyl-pyridine-2-carboxylic acid ethyl ester (Intermediate 1) and commercially available 2-amino-5-cyanopyridine according to the general method of Example 3.

### Example 7

### 4-Ethynyl-6-methyl-pyridine-2-carboxylic acid (1-methyl-1H-pyrazol-3-yl)-amide

### Step A

To a stirred mixture of 4-bromo-6-methyl-pyridine-2-carboxylic acid (1-methyl-1H-pyrazol-3-yl)-amide [CAS No. 947179-05-7] (0.13 g, 0.44 mmol) and triethylamine (0.18 ml, 1.32 mmol)) in THF (2 ml) was added at room temperature and under argon atmosphere triphenylphosphine (3 mg, 0.011 mmol) and PdCl2(PPh3)2 (15 mg, 0.02 mmol) and the mixture was allowed to stir for 30 minutes. Afterwards copper(I) iodide (3 mg, 0.015 mmol) and ethynyltrimethylsilane (0.09 ml, 0.66 mmol) were added. The mixture was allowed to stir for 17 h at room temperature, poured into water (50 ml) and extracted with ethyl acetate (2×50 ml). The combined organic layers were washed with brine (50 ml), dried (MgSO₄) and evaporated to give the crude product as a brown solid (0.22 g), which was further purified by flash chromatography on silica gel [heptan/EtOAc (20-80%)] to yield 6-methyl-4-trimethylsilanylethynyl-pyridine-2-carboxylic acid (1-methyl-1H-pyrazol-3-yl)-amide (130 mg, 94%) as an orange oil, MS (ISP) m/e=313.3 [(M+H)⁺].

### Step B

To a cooled (ice bath) and stirred solution of 6-methyl-4-trimethylsilanylethynyl-pyridine-2-carboxylic acid (1-methyl-1H-pyrazol-3-yl)-amide (130 mg, 0.42 mmol) in methanol (2 ml) and THF (2 ml) was added potassium carbonate (6 mg, 0.043 mmol) was added. The reaction mixture was allowed to stir for 1.5 h at 0° C., poured into water (30 ml) and extracted with ethyl acetate (2×50 ml). The combined organic layers were washed with brine (50 ml), dried (MgSO₄) and evaporated to give the crude product as a light brown solid, which was further purified by flash chromatography on silica gel [heptan/EtOAc (20-80%)] to yield the title compound as a light brown solid (40 mg, 40%), MS (ISP) m/e=241.2 [(M+H)⁺], mp 226.5° C.

### Example 8

### 4-Bromo-6-methyl-pyridine-2-carboxylic acid (5-fluoro-pyridin-2-yl)-amide

The title compound, light yellow solid, MS (ISP) m/e=310.2 [(M+H)⁺], mp 160° C., was prepared from 4-bromo-6-methyl-pyridine-2-carboxylic acid ethyl ester [CAS No. 947179-03-5] and commercially available 2-amino-5-fluoropyridine according to the general method of Example 3.

### Example 9

### 4-Cyano-6-methyl-pyridine-2-carboxylic acid (5-chloro-pyridin-2-yl)-amide

The title compound, light yellow solid, MS (ISP) m/e=273.2 [(M+H)⁺], mp 233.5° C., was prepared from 4-cyano-6-methyl-pyridine-2-carboxylic acid ethyl ester (Intermediate 1) and commercially available 2-amino-5-chloropyridine according to the general method of Example 3.

### Example 10

### 4-Bromo-6-methyl-pyridine-2-carboxylic acid (5-fluoro-6-methyl-pyridin-2-yl)-amide

The title compound, light yellow solid, MS (ISP) m/e=324.2 [(M+H)⁺], mp 181.5° C., was prepared from 4-bromo-6-methyl-pyridine-2-carboxylic acid ethyl ester [CAS No. 947179-03-5] and commercially available 2-amino-5-fluoro-6-methylpyridine according to the general method of Example 3.

### Example 11

### 4-Bromo-6-methyl-pyridine-2-carboxylic acid (5-fluoro-6-methyl-pyridin-2-yl)-amide

The title compound, white solid, MS (ISP) m/e=271.3 [(M+H)⁺], mp 222° C., was prepared in accordance with the general method of Intermediate 1 from 4-bromo-6-methyl-pyridine-2-carboxylic acid (5-fluoro-6-methyl-pyridin-2-yl)-amide (Example 12).

### Example 12

### 4-Cyano-3,6-dimethyl-pyridine-2-carboxylic acid (1-methyl-1H-pyrazol-3-yl)-amide

The title compound, white solid, MS (ISP) m/e=256.3 [(M+H)⁺], mp 180.5° C., was prepared in accordance with the general method of Intermediate 1 from 4-bromo-3,6-dimethyl-pyridine-2-carboxylic acid (1-methyl-1H-pyrazol-3-yl)-amide [CAS No. 947179-44-4].

### Example 13

### 4-Cyano-6-isopropyl-pyridine-2-carboxylic acid (1-methyl-1H-pyrazol-3-yl)-amide

### Step A

4-Bromo-6-isopropyl-pyridine-2-carboxylic acid (1-methyl-1H-pyrazol-3-yl)-amide, white solid, MS (ISP) m/e=325.1 [(M+H)⁺], mp 125° C., was prepared from 4-bromo-6-isopropyl-pyridine-2-carboxylic acid ethyl ester (Intermediate 2) and commercially available 1-methyl-1H-pyrazol-3-yl-amine according to the general method of Example 3.

### Step B

The title compound, white solid, MS (ISP) m/e=270.4 [(M+H)⁺], mp 174° C., was prepared in accordance with the general method of Intermediate 1 from 4-bromo-6-isopropyl-pyridine-2-carboxylic acid (1-methyl-1H-pyrazol-3-yl)-amide.

### Example 14

### 4-Cyano-6-isopropyl-pyridine-2-carboxylic acid (5-fluoro-pyridin-2-yl)-amide

### Step A

4-Bromo-6-isopropyl-pyridine-2-carboxylic acid (5-fluoro-pyridin-2-yl)-amide, white solid, MS (ISP) m/e=340.0 [(M+H)⁺], mp 111° C., was prepared from 4-bromo-6-isopropylpyridine-2-carboxylic acid ethyl ester (Intermediate 2) and commercially available 2-amino-5-fluoropyridine according to the general method of Example 3.

### Step B

The title compound, white solid, MS (ISP) m/e=285.2 [(M+H)⁺], mp 194.5° C., was prepared in accordance with the general method of Intermediate 1 from 4-bromo-6-isopropylpyridine-2-carboxylic acid (5-fluoro-pyridin-2-yl)-amide.

### Example 15

### 4-Cyano-6-trifluoromethyl-pyridine-2-carboxylic acid (1-methyl-1H-pyrazol-3-yl)-amide

### Step A

4-Bromo-6-trifluoromethyl-pyridine-2-carboxylic acid (1-methyl-1H-pyrazol-3-yl)-amide, light yellow solid, MS (ISP) m/e=351.2 [(M+H)⁺], mp 146.5° C., was prepared from 4-bromo-6-trifluoromethyl-pyridine-2-carboxylic acid ethyl ester (Intermediate 3) and commercially available 1-methyl-1H-pyrazol-3-yl-amine according to the general method of Example 3.

### Step B

The title compound, white solid, MS (ISP) m/e=296.3 [(M+H)⁺], mp 190.5° C., was prepared in accordance with the general method of Intermediate 1 from 4-bromo-6-trifluoromethyl-pyridine-2-carboxylic acid (1-methyl-1H-pyrazol-3-yl)-amide.

### Example 16

### 4-Cyano-6-trifluoromethyl-pyridine-2-carboxylic acid (5-fluoro-pyridin-2-yl)-amide

### Step A

4-Bromo-6-trifluoromethyl-pyridine-2-carboxylic acid (5-fluoro-pyridin-2-yl)-amide, yellow solid, MS (ISP) m/e=366.1 [(M+H)⁺], mp 135.5° C., was prepared from 4-bromo-6-trifluoromethyl-pyridine-2-carboxylic acid ethyl ester (Intermediate 3) and commercially available 2-amino-5-fluoropyridine according to the general method of Example 3.

### Step B

The title compound, light yellow solid, MS (ISP) m/e=311.3 [(M+H)⁺], mp 186.5° C., was prepared in accordance with the general method of Intermediate 1 from 4-bromo-6-trifluoromethyl-pyridine-2-carboxylic acid (5-fluoro-pyridin-2-yl)-amide.

### Example 17

### 4-Chloro-6-methyl-pyridine-2-carboxylic acid (5-chloro-pyridin-2-yl)-amide

The title compound, off-white solid, MS (ISP) m/e=282.3 [(M+H)⁺], mp 151° C., was prepared from 4-chloro-6-methyl-pyridine-2-carboxylic acid ethyl ester [CAS-No. 315494-03-2] and commercially available 2-amino-5-chloropyridine according to the general method of Example 3.

### Example 18

### 4-Chloro-6-methyl-pyridine-2-carboxylic acid (5-fluoro-pyridin-2-yl)-amide

The title compound, off-white solid, MS (ISP) m/e=266.1 [(M+H)⁺], mp 156° C., was prepared from 4-chloro-6-methyl-pyridine-2-carboxylic acid ethyl ester [CAS-No. 315494-03-2] and commercially available 2-amino-5-fluoropyridine according to the general method of Example 3.

### Example 19

### 4-Chloro-6-methyl-pyridine-2-carboxylic acid (5-methyl-pyridin-2-yl)-amide

The title compound, off-white solid, MS (ISP) m/e=262.1 [(M+H)⁺], mp 159° C., was prepared from 4-chloro-6-methyl-pyridine-2-carboxylic acid ethyl ester [CAS-No. 315494-03-2] and commercially available 6-amino-3-picoline according to the general method of Example 3.

### Example 20

### 4-Chloro-6-methyl-pyridine-2-carboxylic acid (4-methyl-thiazol-2-yl)-amide

The title compound, off-white solid, MS (ISP) m/e=268.1 [(M+H)⁺], mp 176° C., was prepared from 4-chloro-6-methyl-pyridine-2-carboxylic acid ethyl ester [CAS-No. 315494-03-2] and commercially available 2-amino-4-methylthiazole according to the general method of Example 3.

### Example 21

### 4-Bromo-6-ethyl-pyridine-2-carboxylic acid (5-fluoro-pyridin-2-yl)-amide

The title compound, white solid, MS (ISP) m/e=326.2 [(M+H)⁺], mp 123.5° C., was prepared from 4-bromo-6-ethyl-pyridine-2-carboxylic acid ethyl ester (Intermediate 4) and 2-amino-5-fluoropyridine according to the general method of Example 3.

### Example 22

### 4-Cyano-6-methyl-pyridine-2-carboxylic acid (2-chloro-pyridin-4-yl)-amide

The title compound, light yellow solid, MS (ISP) m/e=273.2 [(M+H)⁺], mp 238.5° C., was prepared from 4-cyano-6-methyl-pyridine-2-carboxylic acid ethyl ester (Intermediate 1) and commercially available 4-amino-2-chloropyridine according to the general method of Example 3.

### Example 23

### 4-Cyano-6-methyl-pyridine-2-carboxylic acid (6-chloro-pyridin-2-yl)-amide

The title compound, light yellow solid, MS (ISP) m/e=273.1 [(M+H)⁺], mp 222.5° C., was prepared from 4-cyano-6-methyl-pyridine-2-carboxylic acid ethyl ester (Intermediate 1) and commercially available 2-amino-6-chloropyridine according to the general method of Example 3.

### Example 24

### 4-Cyano-6-ethyl-pyridine-2-carboxylic acid (1-methyl-1H-pyrazol-3-yl)-amide

### Step A

4-Bromo-6-ethyl-pyridine-2-carboxylic acid (1-methyl-1H-pyrazol-3-yl)-amide, white solid, MS (ISP) m/e=311.2 [(M+H)⁺], mp 135° C., was prepared from 4-bromo-6-ethyl-pyridine-2-carboxylic acid ethyl ester (Intermediate 4) and commercially available 1-methyl-1H-pyrazol-3-yl-amine according to the general method of Example 3.

### Step B

The title compound, off-white solid, MS (ISP) m/e=256.3 [(M+H)⁺], mp 164.5° C., was prepared in accordance with the general method of Intermediate 1 from 4-bromo-6-ethyl-pyridine-2-carboxylic acid (1-methyl-1H-pyrazol-3-yl)-amide.

### Example 25

### 4-Cyano-6-ethyl-pyridine-2-carboxylic acid (5-fluoro-pyridin-2-yl)-amide

The title compound, light yellow solid, MS (ISP) m/e=271.3 [(M+H)⁺], mp 183.5° C., was prepared in accordance with the general method of Intermediate 1 from 4-bromo-6-ethyl-pyridine-2-carboxylic acid (5-fluoro-pyridin-2-yl)-amide (Example 23).

### Example 26

### 4-Cyano-6-methyl-pyridine-2-carboxylic acid (3-chloro-phenyl)-amide

The title compound, white solid, MS (ISP) m/e=272.3 [(M+H)⁺], mp 147.5° C., was prepared from 4-cyano-6-methyl-pyridine-2-carboxylic acid ethyl ester (Intermediate 1) and commercially available 3-chloro-aniline according to the general method of Example 3.

### Example 27

### 4-Cyano-3,6-dimethyl-pyridine-2-carboxylic acid (5-fluoro-pyridin-2-yl)-amide

### Step A

4-Bromo-3,6-dimethyl-pyridine-2-carboxylic acid (5-fluoro-pyridin-2-yl)-amide, white solid, MS (ISP) m/e=324.2 [(M+H)⁺], mp 152.5° C., was prepared from 4-bromo-3,6-dimethyl-pyridine-2-carboxylic acid ethyl ester [CAS No. 947179-26-2] and commercially available 2-amino-5-fluoro-pyridine according to the general method of Example 3.

### Step B

The title compound, white solid, MS (ISP) m/e=271.3 [(M+H)⁺], mp 196.5° C., was prepared in accordance with the general method of Intermediate 1 from 4-bromo-3,6-dimethyl-pyridine-2-carboxylic acid (5-fluoro-pyridin-2-yl)-amide (Example 28).

### Example 28

### 4-Cyano-3,6-dimethyl-pyridine-2-carboxylic acid (5-fluoro-6-methyl-pyridin-2-yl)-amide

### Step A

4-Bromo-3,6-dimethyl-pyridine-2-carboxylic acid (5-fluoro-6-methyl-pyridin-2-yl)-amide, white solid, MS (ISP) m/e=340.1 [(M+H)⁺], mp 160° C., was prepared from 4-bromo-3,6-dimethyl-pyridine-2-carboxylic acid ethyl ester [CAS No. 947179-26-2] and commercially available 2-amino-5-fluoro-6-methyl-pyridine according to the general method of Example 3.

### Step B

The title compound, white solid, MS (ISP) m/e=285.2 [(M+H)⁺], mp 201° C., was prepared in accordance with the general method of Intermediate 1 from 4-bromo-3,6-dimethyl-pyridine-2-carboxylic acid (5-fluoro-6-methyl-pyridin-2-yl)-amide.

### Example 29

### 4-Cyano-3,6-dimethyl-pyridine-2-carboxylic acid (5-chloro-pyridin-2-yl)-amide

The title compound, white solid, MS (ISP) m/e=287.0 [(M+H)⁺], mp 264° C., was prepared from 4-cyano-3,6-dimethyl-pyridine-2-carboxylic acid ethyl ester (Intermediate 5) and commercially available 2-amino-5-chloro-pyridine according to the general method of Example 3.

### Example 30

### 4-Cyano-3,6-dimethyl-pyridine-2-carboxylic acid (4-methyl-thiazol-2-yl)-amide

The title compound, light yellow solid, MS (ISP) m/e=273.2 [(M+H)⁺], mp 199.5° C., was prepared from 4-cyano-3,6-dimethyl-pyridine-2-carboxylic acid ethyl ester (Intermediate 5) and commercially available 2-amino-4-methylthiazole according to the general method of Example 3.

### Example 31

### 4-Cyano-3,6-dimethyl-pyridine-2-carboxylic acid (5-cyano-pyridin-2-yl)-amide

### Step A

4-Bromo-3,6-dimethyl-pyridine-2-carboxylic acid (5-cyano-pyridin-2-yl)-amide, white solid, MS (ISP) m/e=333.1 [(M+H)⁺], mp 225° C., was prepared from 4-bromo-3,6-dimethyl-pyridine-2-carboxylic acid ethyl ester [CAS No. 947179-26-2] and commercially available 2-amino-5-cyano-pyridine according to the general method of Example 3.

### Step B

The title compound, white solid, MS (ISP) m/e=278.2 [(M+H)⁺], mp 216° C., was prepared in accordance with the general method of Intermediate 1 from 4-bromo-3,6-dimethyl-pyridine-2-carboxylic acid (5-cyano-pyridin-2-yl)-amide.

### Example 32

### 4-Cyano-3,6-dimethyl-pyridine-2-carboxylic acid (5-methyl-pyridin-2-yl)-amide

### Step A

4-Bromo-3,6-dimethyl-pyridine-2-carboxylic acid (5-methyl-pyridin-2-yl)-amide, off-white solid, MS (ISP) m/e=322.1 [(M+H)⁺], mp 160.5° C., was prepared from 4-bromo-3,6-dimethyl-pyridine-2-carboxylic acid ethyl ester [CAS No. 947179-26-2] and commercially available 2-amino-5-methyl-pyridine according to the general method of Example 3.

### Step B

The title compound, white solid, MS (ISP) m/e=267.2 [(M+H)⁺], mp 212.5° C., was prepared in accordance with the general method of Intermediate 1 from 4-bromo-3,6-dimethyl-pyridine-2-carboxylic acid (5-methyl-pyridin-2-yl)-amide.

### Example 33

### 4-Cyano-6-trifluoromethyl-pyridine-2-carboxylic acid (5-chloro-pyridin-2-yl)-amide

The title compound, off-white solid, MS (ISP) m/e=327.1 [(M+H)⁺], mp 208° C., was prepared from 4-cyano-6-trifluoromethyl-pyridine-2-carboxylic acid ethyl ester (Intermediate 6) and commercially available 2-amino-5-chloro-pyridine according to the general method of Example 3.

### Example 34

### 4-Chloro-6-methyl-pyridine-2-carboxylic acid (2-chloro-pyridin-4-yl)-amide

The title compound, white solid, MS (ISP) m/e=282.1 [(M+H)⁺], mp 221° C., was prepared from 4-chloro-6-methyl-pyridine-2-carboxylic acid ethyl ester [CAS-No. 315494-03-2] and commercially available 4-amino-2-chloropyridine according to the general method of Example 3.

### Example 35

### 4-Chloro-6-methyl-pyridine-2-carboxylic acid (6-chloro-pyridin-2-yl)-amide

The title compound, white solid, MS (ISP) m/e=282.1 [(M+H)⁺], mp 176° C., was prepared from 4-chloro-6-methyl-pyridine-2-carboxylic acid ethyl ester [CAS-No. 315494-03-2] and commercially available 2-amino-6-chloropyridine according to the general method of Example 3.

### Example 36

### 4-Chloro-6-methyl-pyridine-2-carboxylic acid (6-methyl-pyridin-2-yl)-amide

The title compound, white solid, MS (ISP) m/e=262.1 [(M+H)⁺], mp 130.5° C., was prepared from 4-chloro-6-methyl-pyridine-2-carboxylic acid ethyl ester [CAS-No. 315494-03-2] and commercially available 6-amino-2-picoline according to the general method of Example 3.

### Example 37

### 4-Chloro-6-methyl-pyridine-2-carboxylic acid (3-chloro-phenyl)-amide

The title compound, white solid, MS (ISP) m/e=281.0 [(M+H)⁺], mp 174° C., was prepared from 4-chloro-6-methyl-pyridine-2-carboxylic acid ethyl ester [CAS-No. 315494-03-2] and commercially available 3-chloroaniline according to the general method of Example 3.

### Example 38

### 4-Cyano-6-methyl-pyridine-2-carboxylic acid (3-chloro-4-fluoro-phenyl)-amide

The title compound, white solid, MS (ISP) m/e=290.1 [(M+H)⁺], mp 204° C., was prepared from 4-cyano-6-methyl-pyridine-2-carboxylic acid ethyl ester (Intermediate 1) and commercially available 3-chloro-4-fluoro-aniline according to the general method of Example 3.

### Example 39

### 4-Cyano-6-methyl-pyridine-2-carboxylic acid (4-fluoro-phenyl)-amide

The title compound, off-white solid, MS (ISP) m/e=256.2 [(M+H)⁺], mp 189° C., was prepared from 4-cyano-6-methyl-pyridine-2-carboxylic acid ethyl ester (Intermediate 1) and commercially available 4-fluoro-aniline according to the general method of Example 3.

### Example 40

### 4-Cyano-6-hydroxymethyl-pyridine-2-carboxylic acid (3-chloro-phenyl)-amide

The title compound, light brown solid, MS (ISP) m/e=288.1 [(M+H)⁺], mp 167° C., was prepared from 4-cyano-6-hydroxymethyl-pyridine-2-carboxylic acid ethyl ester (Intermediate 7) and commercially available 3-chloro-aniline according to the general method of Example 3.

### Example 41

### 4-Cyano-6-methyl-pyridine-2-carboxylic acid (3-fluoro-phenyl)-amide

The title compound, white solid, MS (ISP) m/e=256.2 [(M+H)⁺], mp 139° C., was prepared from 4-cyano-6-methyl-pyridine-2-carboxylic acid ethyl ester (Intermediate 1) and commercially available 3-fluoro-aniline according to the general method of Example 3.

### Example 42

### 4-Cyano-6-methyl-pyridine-2-carboxylic acid (3,4-difluoro-phenyl)-amide

The title compound, white solid, MS (ISP) m/e=274.2 [(M+H)⁺], mp 170° C., was prepared from 4-cyano-6-methyl-pyridine-2-carboxylic acid ethyl ester (Intermediate 1) and commercially available 3,4-difluoro-aniline according to the general method of Example 3.

### Example 43

### 4-Cyano-6-methyl-pyridine-2-carboxylic acid m-tolylamide

The title compound, white solid, MS (ISP) m/e=252.2 [(M+H)⁺], mp 160° C., was prepared from 4-cyano-6-methyl-pyridine-2-carboxylic acid ethyl ester (Intermediate 1) and commercially available 3-methyl-aniline according to the general method of Example 3.

### Example 44

### 4-Cyano-6-methyl-pyridine-2-carboxylic acid (3-trifluoromethyl-phenyl)-amide

The title compound, white solid, MS (ISP) m/e=306.1 [(M+H)⁺], mp 167° C., was prepared from 4-cyano-6-methyl-pyridine-2-carboxylic acid ethyl ester (Intermediate 1) and commercially available 3-trifluoromethyl-aniline according to the general method of Example 3.

### Example 45

### 4-Cyano-6-methyl-pyridine-2-carboxylic acid (3-methoxy-phenyl)-amide

The title compound, off-white solid, MS (ISP) m/e=268.2 [(M+H)⁺], mp 189° C., was prepared from 4-cyano-6-methyl-pyridine-2-carboxylic acid ethyl ester (Intermediate 1) and commercially available 3-methoxy-aniline according to the general method of Example 3.

### Example 46

### 4-Cyano-6-hydroxymethyl-pyridine-2-carboxylic acid (5-fluoro-pyridin-2-yl)-amide

The title compound, light yellow solid, MS (ISP) m/e=273.2 [(M+H)⁺], mp 232.5° C., was prepared from 4-cyano-6-hydroxymethyl-pyridine-2-carboxylic acid ethyl ester (Intermediate 7) and commercially available 2-amino-5-fluoro-pyridine according to the general method of Example 3.

### Example 47

### 4-Cyano-6-methyl-pyridine-2-carboxylic acid (6-methyl-pyridin-2-yl)-amide

The title compound, white solid, MS (ISP) m/e=253.1 [(M+H)⁺], mp 243.5° C., was prepared from 4-cyano-6-methyl-pyridine-2-carboxylic acid ethyl ester (Intermediate 1) and commercially available 2-amino-6-methyl-pyridine according to the general method of Example 3.

### Example 48

### 4-Cyano-6-methyl-pyridine-2-carboxylic acid (2-fluoro-pyridin-4-yl)-amide

The title compound, off-white solid, MS (ISP) m/e=257.2 [(M+H)⁺], mp 246.5° C., was prepared from 4-cyano-6-methyl-pyridine-2-carboxylic acid ethyl ester (Intermediate 1) and commercially available 3-amino-2-fluoro-pyridine according to the general method of Example 3.

### Example 49

### 4-Cyano-6-methyl-pyridine-2-carboxylic acid (4-fluoro-3-methyl-phenyl)-amide

The title compound, off-white solid, MS (ISP) m/e=270.2 [(M+H)⁺], mp 177° C., was prepared from 4-cyano-6-methyl-pyridine-2-carboxylic acid ethyl ester (Intermediate 1) and commercially available 4-fluoro-3-methylaniline according to the general method of Example 3.

### Example 50

### 4-Cyano-6-methyl-pyridine-2-carboxylic acid (2-methyl-pyridin-4-yl)-amide

The title compound, off-white solid, MS (ISP) m/e=253.1 [(M+H)⁺], mp 302° C., was prepared from 4-cyano-6-methyl-pyridine-2-carboxylic acid ethyl ester (Intermediate 1) and commercially available 4-amino-2-methyl-pyridine according to the general method of Example 3.

### Example 51

### 4-Chloro-6-methyl-pyridine-2-carboxylic acid m-tolylamide

The title compound, white solid, MS (ISP) m/e=261.1 [(M+H)⁺], mp 95° C., was prepared from 4-chloro-6-methyl-pyridine-2-carboxylic acid ethyl ester [CAS-No. 315494-03-2] and commercially available 3-methylaniline according to the general method of Example 3.

### Example 52

### 4-Chloro-6-methyl-pyridine-2-carboxylic acid (3-fluoro-phenyl)-amide

The title compound, white solid, MS (ISP) m/e=265.0 [(M+H)⁺], mp 123° C., was prepared from 4-chloro-6-methyl-pyridine-2-carboxylic acid ethyl ester [CAS-No. 315494-03-2] and commercially available 3-fluoroaniline according to the general method of Example 3.

### Example 53

### 4-Chloro-6-methyl-pyridine-2-carboxylic acid (2-methyl-pyridin-4-yl)-amide

The title compound, white solid, MS (ISP) m/e=262.1 [(M+H)⁺], mp 162° C., was prepared from 4-chloro-6-methyl-pyridine-2-carboxylic acid ethyl ester [CAS-No. 315494-03-2] and commercially available 4-amino-2-methyl-pyridine according to the general method of Example 3.

1. A compound of formula (I) 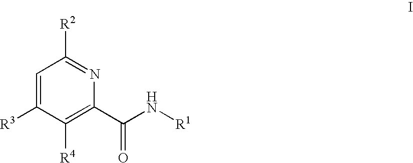
wherein
R¹ is an aromatic 5- or 6-membered ring selected from the group consisting of 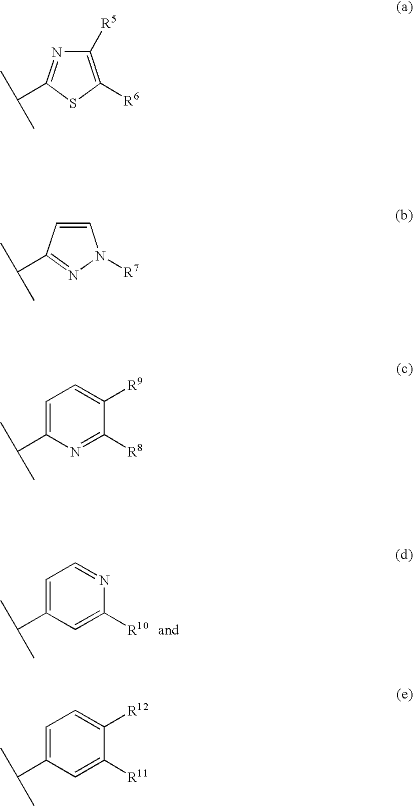
R² is C₁-C₃-alkyl, optionally substituted with one or more OH or halo;
R³ is halo, cyano, or ethynyl;
R⁴ is H or C₁-C₃-alkyl; and
R⁵, R⁶, R⁷, R⁸, R⁹, R¹⁰, R¹¹ and R¹² are each independently H, halo, CN, C₁-C₃-alkyl, C₁-C₃-haloalkyl, C₁-C₃-alkoxy, C₁-C₃-haloalkoxy or OH;
or a pharmaceutically acceptable salt thereof.

2. The compound of claim 1, wherein R¹ is n aromatic 5- or 6-membered ring selected from the group consisting of 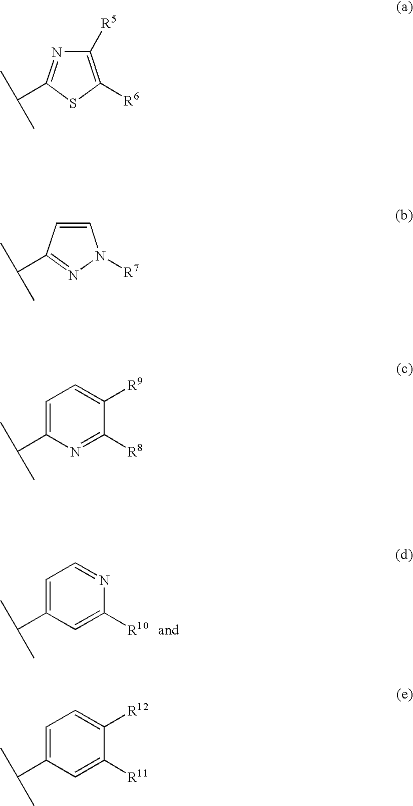
and R⁵, R⁶, R⁷, R⁸, R⁹, R¹⁰, R¹¹ and R¹² are each independently H, halo, CN, C₁-C₃-alkyl, C₁-C₃-fluoroalkyl, C₁-C₃-alkoxy, C₁-C₃-fluoroalkoxy or OH.

3. The compound of claim 2, wherein R⁵, R⁶, R⁷, R⁸, R⁹, R¹⁰, R¹¹ and R¹² are each independently H, fluoro, chloro, cyano, methyl, trifluoromethyl or methoxy.

4. The compound of claim 1, wherein R² is methyl, ethyl, i-propyl, hydroxymethyl or trifluoromethyl.

5. The compound of claim 1, wherein R³ is chloro, bromo, cyano or ethynyl.

6. The compound of claim 1, wherein R⁴ is H.

7. The compound of claim 1, selected from the group consisting of
4-Cyano-6-methyl-pyridine-2-carboxylic acid (4-methyl-thiazol-2-yl)-amide,
4-Chloro-6-methyl-pyridine-2-carboxylic acid (4-methyl-thiazol-2-yl)-amide,
4-Cyano-3,6-dimethyl-pyridine-2-carboxylic acid (4-methyl-thiazol-2-yl)-amide,
4-Cyano-6-methyl-pyridine-2-carboxylic acid (1-methyl-1H-pyrazol-3-yl)-amide,
4-Chloro-6-methyl-pyridine-2-carboxylic acid (1-methyl-1H-pyrazol-3-yl)-amide,
4-Ethynyl-6-methyl-pyridine-2-carboxylic acid (1-methyl-1H-pyrazol-3-yl)-amide,
4-Cyano-3,6-dimethyl-pyridine-2-carboxylic acid (1-methyl-1H-pyrazol-3-yl)-amide,
4-Cyano-6-ethyl-pyridine-2-carboxylic acid (1-methyl-1H-pyrazol-3-yl)-amide,
4-Cyano-6-methyl-pyridine-2-carboxylic acid (5-fluoro-pyridin-2-yl)-amide, and
4-Cyano-6-methyl-pyridine-2-carboxylic acid (5-methyl-pyridin-2-yl)-amide.

8. The compound of claim 1, selected from the group consisting of
4-Cyano-6-methyl-pyridine-2-carboxylic acid (5-cyano-pyridin-2-yl)-amide,
4-Bromo-6-methyl-pyridine-2-carboxylic acid (5-fluoro-pyridin-2-yl)-amide,
4-Cyano-6-methyl-pyridine-2-carboxylic acid (5-chloro-pyridin-2-yl)-amide,
4-Bromo-6-methyl-pyridine-2-carboxylic acid (5-fluoro-6-methyl-pyridin-2-yl)-amide,
4-Cyano-6-methyl-pyridine-2-carboxylic acid (5-fluoro-6-methyl-pyridin-2-yl)-amide,
4-Cyano-6-trifluoromethyl-pyridine-2-carboxylic acid (5-fluoro-pyridin-2-yl)-amide,
4-Chloro-6-methyl-pyridine-2-carboxylic acid (5-fluoro-pyridin-2-yl)-amide,
4-Chloro-6-methyl-pyridine-2-carboxylic acid (5-methyl-pyridin-2-yl)-amide,
4-Cyano-6-methyl-pyridine-2-carboxylic acid (6-chloro-pyridin-2-yl)-amide, and
4-Cyano-6-ethyl-pyridine-2-carboxylic acid (5-fluoro-pyridin-2-yl)-amide.

9. The compound of claim 1, selected from the group consisting of
4-Cyano-3,6-dimethyl-pyridine-2-carboxylic acid (5-fluoro-pyridin-2-yl)-amide,
4-Cyano-3,6-dimethyl-pyridine-2-carboxylic acid (5-chloro-pyridin-2-yl)-amide,
4-Chloro-6-methyl-pyridine-2-carboxylic acid (6-chloro-pyridin-2-yl)-amide,
4-Chloro-6-methyl-pyridine-2-carboxylic acid (6-methyl-pyridin-2-yl)-amide,
4-Cyano-6-hydroxymethyl-pyridine-2-carboxylic acid (5-fluoro-pyridin-2-yl)-amide,
4-Cyano-6-methyl-pyridine-2-carboxylic acid (6-methyl-pyridin-2-yl)-amide,
4-Cyano-6-methyl-pyridine-2-carboxylic acid (2-chloro-pyridin-4-yl)-amide,
4-Cyano-6-methyl-pyridine-2-carboxylic acid (2-fluoro-pyridin-4-yl)-amide,
4-Cyano-6-methyl-pyridine-2-carboxylic acid (2-methyl-pyridin-4-yl)-amide, and
4-Cyano-6-methyl-pyridine-2-carboxylic acid (3-chloro-phenyl)-amide.

10. The compound of claim 1, selected from the group consisting of
4-Chloro-6-methyl-pyridine-2-carboxylic acid (3-chloro-phenyl)-amide,
4-Cyano-6-methyl-pyridine-2-carboxylic acid (3-chloro-4-fluoro-phenyl)-amide,
4-Cyano-6-hydroxymethyl-pyridine-2-carboxylic acid (3-chloro-phenyl)-amide,
4-Cyano-6-methyl-pyridine-2-carboxylic acid (3-fluoro-phenyl)-amide,
4-Cyano-6-methyl-pyridine-2-carboxylic acid (3,4-difluoro-phenyl)-amide,
4-Cyano-6-methyl-pyridine-2-carboxylic acid m-tolylamide,
4-Cyano-6-methyl-pyridine-2-carboxylic acid (3-trifluoromethyl-phenyl)-amide,
4-Cyano-6-methyl-pyridine-2-carboxylic acid (3-methoxy-phenyl)-amide,
4-Cyano-6-methyl-pyridine-2-carboxylic acid (4-fluoro-3-methyl-phenyl)-amide, and
4-Chloro-6-methyl-pyridine-2-carboxylic acid m-tolylamide.

11. A pharmaceutical composition comprising a therapeutically effective amount of a compound of formula I 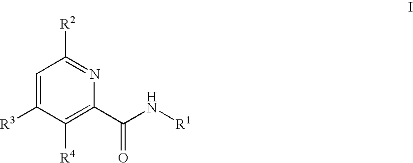
wherein
R¹ is an aromatic 5- or 6-membered ring selected from the group consisting of 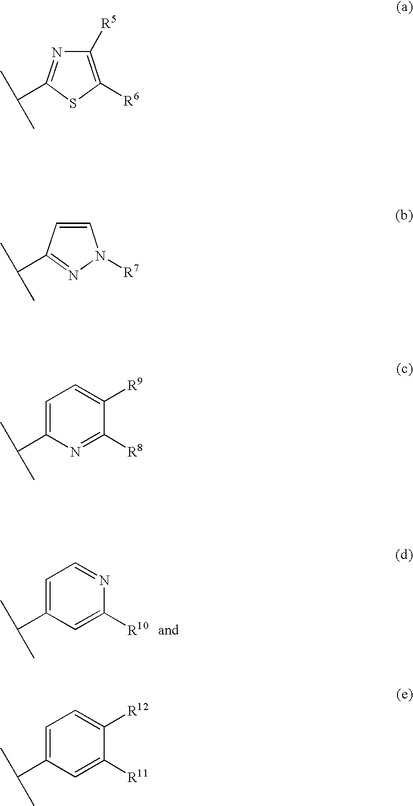
R² is C₁-C₃-alkyl, optionally substituted with one or more OH or halo;
R³ is halo, cyano, or ethynyl;
R⁴ is H or C₁-C₃-alkyl; and
R⁵, R⁶, R⁷, R⁸, R⁹, R¹⁰, R¹¹ and R¹² are each independently H, halo, CN, C₁-C₃-alkyl, C₁-C₃-haloalkyl, C₁-C₃-alkoxy, C₁-C₃-haloalkoxy or OH;
or a pharmaceutically acceptable salt thereof and a pharmaceutically acceptable carrier.
